(19) United States
(12) Patent Application Publication
(10) Pub. No.: US 2014/0221778 A1
(43) Pub. Date: Aug. 7, 2014

(54) Identifying Physiological Parameters from Raw Data Received Wirelessly from a Sensor

(71) Applicants: Abraham Carter, Salt Lake City, UT (US); David Scott, Salt Lake City, UT (US)

(72) Inventors: Abraham Carter, Salt Lake City, UT (US); David Scott, Salt Lake City, UT (US)

(21) Appl. No.: 14/172,726

(22) Filed: Feb. 4, 2014

Related U.S. Application Data
(60) Provisional application No. 61/761,635, filed on Feb. 6, 2013.

(51) Int. Cl. A61B 5/00; A61B 5/145; A61B 5/11; A61B 5/1455

(52) U.S. Cl. CPC A61B 5/0022; A61B 5/14551; A61B 5/14532; A61B 5/1118
USPC 600/301

(57) ABSTRACT

The present invention is directed to identifying physiological parameters from raw data received wirelessly from a sensor. The invention allows a user to track the physiological parameters using any of a number of common portable devices, such as a smart phone. In this manner, the user is not required to own, wear, or carry a specialized device for receiving and processing raw data received from the sensors.

### CROSS-REFERENCE TO RELATED APPLICATIONS

This application claims the benefit of U.S. Provisional Application No. 61/761,635 which was filed on Feb. 6, 2013.

### BACKGROUND

Many devices have been developed for tracking various physiological parameters during a user's workout. For example, heart rate monitors can detect a user's heart rate, pulse oximeters can detect the saturation of a user's hemoglobin, blood glucose monitors can detect the glucose level in a user's blood, etc.

To track these various parameters, the user is required to wear or carry a specialized device that can both receive raw data from the sensors and process the raw data to generate useful information displayable to the user. For example, many GPS watches are configured to receive raw data from a heart rate monitor (e.g. via Bluetooth) and convert the raw data into an indication of the user's heart rate. Similarly, other devices are configured to receive raw data from a pulse oximeter attached to the user's finger and convert the raw data into an indication of the hemoglobin saturation level in the user's blood.

The requirement that a specialized device be worn or carried can often discourage a user from using such devices. For example, the user may be unable or unwilling to wear a specialized device at all times, and therefore, may be without the device at a time when he desires to measure various physiological parameters. Also, users are often discouraged by the price and complexity of such devices.

### BRIEF SUMMARY

The present invention extends to methods, systems, and computer program products for identifying physiological parameters from raw data received wirelessly from a sensor. The invention allows a user to track the physiological parameters using any of a number of common portable devices, such as a smart phone. In this manner, the user is not required to own, wear, or carry a specialized device for receiving and processing raw data received from the sensors.

In one embodiment, the present invention is implemented as a method for identifying physiological parameters from raw data received wirelessly from a sensor. The method includes receiving, at a mobile application executing on a mobile phone, raw data generated by one or more sensors being worn by a user. The one or more sensors are configured to detect one or more physiological parameters of the user during an activity. The received raw data is processed by the mobile application to generate usable data representing a measurement of the one or more physiological parameters. The usable data is then displayed by the mobile application such that the user is informed of the measurement of the one or more physiological parameters.

In another embodiment, the present invention is implemented as a system for monitoring physiological parameters during an activity using a mobile phone. The system comprises: a mobile phone having an application for receiving raw sensor data from one or more sensors worn by a user while performing an activity. The one or more sensors detect one or more physiological parameters of the user while the user performs the activity and transmit the raw sensor data to the mobile phone. The mobile phone processes the raw sensor data to generate usable data and to display the usable data on a display of the mobile phone.

This summary is provided to introduce a selection of concepts in a simplified form that are further described below in the Detailed Description. This Summary is not intended to identify key features or essential features of the claimed subject matter.

Additional features and advantages of the invention will be set forth in the description which follows, and in part will be obvious from the description, or may be learned by the practice of the invention. The features and advantages of the invention may be realized and obtained by means of the instruments and combinations particularly pointed out in the appended claims. These and other features of the present invention will become more fully apparent from the following description and appended claims, or may be learned by the practice of the invention as set forth hereinafter.

### BRIEF DESCRIPTION OF THE DRAWINGS

In order to describe the manner in which the above-recited and other advantages and features of the invention can be obtained, a more particular description of the invention briefly described above will be rendered by reference to specific embodiments thereof which are illustrated in the appended drawings. Understanding that these drawings depict only typical embodiments of the invention and are not therefore to be considered to be limiting of its scope, the invention will be described and explained with additional specificity and detail through the use of the accompanying drawings in which:

FIG. 1 illustrates an exemplary computing environment in which the present invention can be implemented; and

FIGS. 2A and 2B illustrate the transmission of raw sensor data between the sensors and the portable computing device of the present invention.

### DETAILED DESCRIPTION

The present invention extends to methods, systems, and computer program products for identifying physiological parameters from raw data received wirelessly from a sensor. The invention allows a user to track the physiological parameters using any of a number of common portable devices, such as a smart phone. In this manner, the user is not required to own, wear, or carry a specialized device for receiving and processing raw data received from the sensors.

In one embodiment, the present invention is implemented as a method for identifying physiological parameters from raw data received wirelessly from a sensor. The method includes receiving, at a mobile application executing on a mobile phone, raw data generated by one or more sensors being worn by a user. The one or more sensors are configured to detect one or more physiological parameters of the user during an activity. The received raw data is processed by the mobile application to generate usable data representing a measurement of the one or more physiological parameters. The usable data is then displayed by the mobile application such that the user is informed of the measurement of the one or more physiological parameters.

In another embodiment, the present invention is implemented as a system for monitoring physiological parameters during an activity using a mobile phone. The system comprises: a mobile phone having an application for receiving raw sensor data from one or more sensors worn by a user while performing an activity. The one or more sensors detect one or more physiological parameters of the user while the user performs the activity and transmit the raw sensor data to the mobile phone. The mobile phone processes the raw sensor data to generate usable data and to display the usable data on a display of the mobile phone.

Embodiments of the present invention may comprise or utilize special purpose or general-purpose computers including computer hardware, such as, for example, one or more processors and system memory, as discussed in greater detail below. Embodiments within the scope of the present invention also include physical and other computer-readable media for carrying or storing computer-executable instructions and/or data structures. Such computer-readable media can be any available media that can be accessed by a general purpose or special purpose computer system.

Computer-readable media is categorized into two disjoint categories: computer storage media and transmission media. Computer storage media (devices) include RAM, ROM, EEPROM, CD-ROM, solid state drives (“SSDs”) (e.g., based on RAM), Flash memory, phase-change memory (“PCM”), other types of memory, other optical disk storage, magnetic disk storage or other magnetic storage devices, or any other similarly storage medium which can be used to store desired program code means in the form of computer-executable instructions or data structures and which can be accessed by a general purpose or special purpose computer. Transmission media include signals and carrier waves.

Computer-executable instructions comprise, for example, instructions and data which, when executed by a processor, cause a general purpose computer, special purpose computer, or special purpose processing device to perform a certain function or group of functions. The computer executable instructions may be, for example, binaries, intermediate format instructions such as assembly language or P-Code, or even source code.

Those skilled in the art will appreciate that the invention may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, tablets, pagers, routers, switches, and the like.

The invention may also be practiced in distributed system environments where local and remote computer systems, which are linked (either by hardwired data links, wireless data links, or by a combination of hardwired and wireless data links) through a network, both perform tasks. In a distributed system environment, program modules may be located in both local and remote memory storage devices. An example of a distributed system environment is a cloud of networked servers or server resources. Accordingly, the present invention can be hosted in a cloud environment.

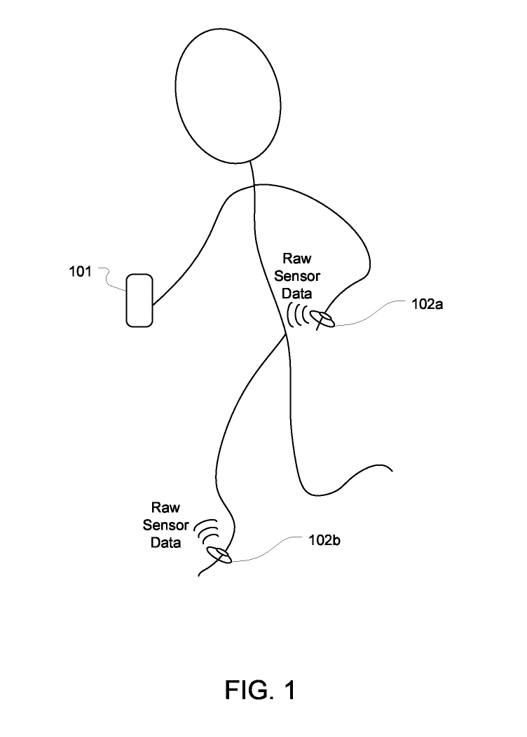

FIG. 1 illustrates an exemplary computer environment 100 in which the present invention can be implemented. Computer environment 100 includes a portable computing device 101 and sensors 102a, 102b that are worn by a user during a workout or other activity. In a typical implementation, portable computing device 101 can be a user's smart phone or other device capable of running a mobile application (e.g. an MP3 player or tablet). Sensors 102a, 102b can represent different types of sensors for detecting various physiological parameters. For example, in a particular embodiment, the sensors can include a blood glucose sensor, a pulse oximeter, a skin temperature sensor, or a blood pressure sensor. The term sensor should be understood as referring to either or both the individual sensor unit and the housing containing the sensor unit.

In some embodiments, in addition to the sensors for detecting various physiological parameters, one or more of the sensors can include an accelerometer that is used to detect specific movements of the user's body parts on which the sensors are worn. For example, in the particular embodiment shown in FIG. 1, sensor 102a is worn around the user's wrist (e.g. as a bracelet) and includes an accelerometer for determining the specific motion the user's arm makes during a workout. In such embodiments, sensor 102a can also include one or more sensors for detecting one or more of the user's physiological parameters during the workout.

Similarly, sensor 102b, as shown in FIG. 1, can be worn on or around the user's foot or ankle and provide accelerometer data representing the specific motion of the user's leg during the workout. Sensor 102b can also contain one or more sensors for detecting various physiological parameters.

Although FIG. 1 depicts two sensors 102a, 102b being worn around the wrist and on the foot respectively, the present invention is not limited to any specific number of sensors or any particular placement of the sensors on the user's body. For example, one or more sensors can be worn on the elbow, hip, knee, head, etc.

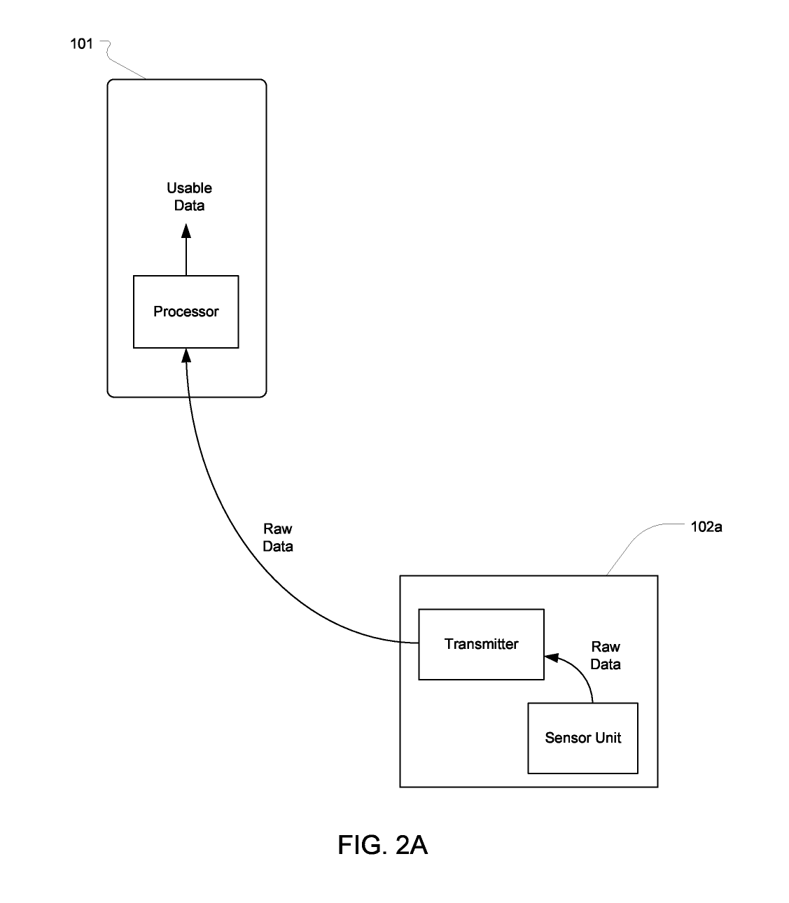

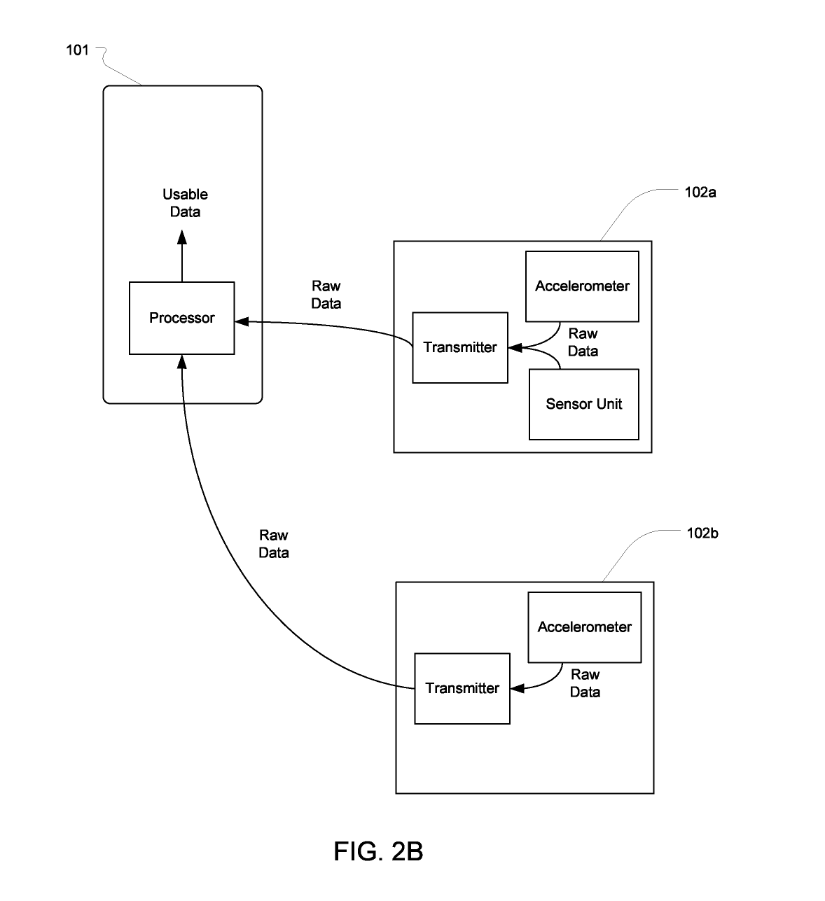

FIGS. 2A and 2B represent how a sensor can transmit raw data to portable computing device 101. Previous approaches process raw data received from a sensor into usable data prior to transmitting the usable data to another computing device. The present invention differs from this approach in that the sensor transmits raw data wirelessly to portable computing device 101. For example, using the techniques of the present invention, raw data from a pulse oximeter or a blood glucose monitor can be wirelessly transmitted directly to the user's smart phone where it is processed to generate usable data. In this way, the user does not need a specialized device, but can use his smart phone or other similar device to track physiological parameters. Because a user generally carries a smart phone or similar device at all times, processing raw data in this way (i.e. by transmitting the raw data directly to the smart phone for processing) allows for a simpler and more accessible system.

As shown in FIG. 2A, sensor 102a includes a sensor unit (e.g. a blood glucose monitor or pulse oximeter). Raw data output by the sensor unit is transmitted directly from sensor 102a to portable computing device 101 where it is processed into usable data. FIG. 2B illustrates that sensor 102a can also include an accelerometer whose raw data is also transmitted to portable computing device 101 where it is processed into usable data. FIG. 2B also shows that sensor 102b may only include an accelerometer. Of course, sensor 102b could also include one or more sensor units for generating raw data related to another physiological parameter which could be wirelessly transmitted to portable computing device 101 for processing.

In some embodiments, sensors 102a, 102b can be configured to intermittently transmit sensor data. This can be done to conserve battery power. For example, sensors 102a, 102b can include logic for determining when the sensor data is of particular importance, and transmit only the important data. In one example, sensors 102a, 102b can be configured to only transmit data when a significant change in the data has occurred. In such cases, portable computing device 101 can assume that the physiological parameter being sensed has not changed significantly until it again receives a transmission from the sensors.

Once portable computing device 101 has received and processed the raw data into usable data, the usable data can be displayed to the user carrying the portable computing device. For example, a mobile application on the user's smart phone can be used to view a measurement of the one or more physiological parameters.

In some embodiments, the raw data can also be transmitted to a server or other computing system where it can be further processed and analyzed. For example, the mobile application on portable computing device 101 can be configured to upload or otherwise transfer raw data to a central server system that stores raw data received from many different portable computing devices. This raw data can be compiled into a repository where further analysis can be performed to identify patterns, trends, tendencies, etc. which can later be provided to portable computing device 101 for automatic and immediate processing of raw data.

For example, if it is determined, after processing a large set of raw data from many different users, that a particular pattern appears in the raw data that can be used as an indicator that a user is performing to a maximum level or may be suffering from a condition, this pattern can be sent back to portable computing device 101. The portable computing device can then automatically scan new raw data being received from the sensors and notify the user if the pattern is detected in the new raw data. Examples of the types of conditions that can be identified in this manner include ideal training levels (e.g. VO₂ max, lactate threshold), over-training, depletion of blood glucose levels, etc.

Identifying that a particular pattern can serve as an indicator of some condition can be performed in any suitable way. Because portable computing device 101 receives the raw sensor data and will generally contain circuitry for transferring the raw data to a central server, the present invention facilitates this analysis. In other words, using the present invention, the raw sensor data (as opposed to processed and possibly proprietary data) can easily be provided to a central repository where it can more easily processed.

This raw data can be mined to identify patterns or neural networks. Correlations can also be created between the raw data and activity data to enable the detection of the early onset of a disease or to enhance patient care monitoring.

FIG. 2B also represents a system that includes multiple accelerometer devices (or sensors) where at least one of the accelerometer devices also includes a sensor for detecting one or more physiological parameters. In this type of system, the user can wear the devices on various body parts (e.g. the wrist and foot). The accelerometers can route raw accelerometer data to portable computing device 101 (e.g. the user's smart phone) which processes the raw accelerometer data to determine specific movements the user is performing during a workout. For example, portable computing device 101 can detect, based on the raw accelerometer data, that the user is riding a bike, running, doing push-ups, pull-ups, curls, etc.

The additional raw data received from the one or more sensors for detecting physiological parameters can also be processed and correlated with the raw accelerometer data to provide additional feedback regarding how the specific movements or exercises are being performed (e.g. by matching time stamps so the appropriate sensor data is used with the accelerometer data generated at the same time as the sensor data). In other words, using this system, the user's portable computing device can track the specific type of exercise being performed (including the number of reps) while at the same time providing feedback regarding how well the user's body is responding by tracking physiological parameters. This tracking and monitoring can all be performed on portable computing device 101.

It is noted again that, although devices exist for detecting some physiological parameters including devices that can be worn during exercise, such devices are specialized for performing such detection and analysis. In contrast, the system of the present invention can employ a standard portable computing device that receives raw sensor data and processes the raw data to generate usable data.

In a specific example, the present invention would allow a user to use an app on his iPhone to track specific physiological parameters (rather than having to purchase a separate device (e.g. a watch or armband) that contains customized hardware/software for processing raw sensor data into a usable form. Accordingly, the present invention provides a simplified system for use during exercise or other activities. Because the sensors can transmit raw data directly to the portable computing device, and because the portable computing device can run a mobile application capable of processing the raw data, virtually anyone can begin tracking physiological parameters by simply wearing one or more sensors.

As another example of how the present invention improves on current systems, many users carry a phone or other audio device (e.g. an iPod) during exercise both as forms of entertainment (e.g. music) and for tracking distance traveled (e.g. via a GPS based application). For such a user to be able to monitor physiological parameters during a workout, the user must purchase a separate device (e.g. a watch or armband) that includes sensors for generating raw data and hardware/software for processing the raw data into a usable form and displaying the usable data. Such devices are expensive and require the user to carry multiple devices (assuming the user desires to also carry his phone).

In contrast, in the present invention, the user only needs to carry his phone because the sensors can transmit the raw data directly to the phone where an app processes the raw data and displays usable data on the phone's display. The phone also contains the necessary circuitry to upload or transfer the raw and/or usable data to another system for further use.

In some embodiments, portable computing device 101 can be configured to communicate wirelessly with sensors 102a, 102b (e.g. via Bluetooth) to update firmware on the sensors. Updating the firmware in this manner enables the sensors to be customized for a particular user based on previously received sensor data.

For example, a sensor may initially contain firmware that causes the sensor to perform in a manner that would be most effective for an average person. However, as the user begins using the sensor, the data generated by the sensor can be analyzed to determine whether changes to the firmware would improve the performance of the sensor. If so, updates to the firmware can be performed directly over a wireless connection between portable computing device 101 and the sensor. In some embodiments, the analysis of whether the sensor's firmware can be updated to improve performance can be performed on portable computing device 101, and even automatically (e.g. as the user is exercising or after a workout).

In one example, the firmware may be configured to cause a sensor to emit a light sufficient to penetrate the skin of a person of average weight. However, if the user is heavier than average (and therefore requires a stronger intensity of light for the sensor to adequately work), the firmware can be adjusted so that a stronger light is emitted. Allowing the dynamic adjustment of firmware in this manner can be beneficial for improving the quality of sensor data as well as to conserve battery power (e.g. by not emitting more light than necessary).

In another example, the firmware can be adjusted based on the amount of movement the user makes. For example, a user that moves relatively little may not need as frequent sensor readings as a user that frequently moves. In such cases, the sensor's firmware can be dynamically updated to control how frequently sensor readings are made to optimize the performance of the sensor (e.g. power efficiency or storage requirement).

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed:

1. A method for identifying physiological parameters from raw data received wirelessly from a sensor, the method comprising:
receiving, at a mobile application executing on a mobile phone, raw data generated by one or more sensors being worn by a user, the one or more sensors being configured to detect one or more physiological parameters of the user during an activity;
processing, by the mobile application, the received raw data to generate usable data representing a measurement of the one or more physiological parameters; and
displaying, by the mobile application, the usable data such that the user is informed of the measurement of the one or more physiological parameters.

2. The method of claim 1, wherein the raw data generated by one or more sensors comprises raw data generated by one or more of a pulse oximeter or a blood glucose monitor.

3. The method of claim 1, wherein the raw data generated by one or more sensors comprises raw data generated by one or more accelerometers.

4. The method of claim 1, further comprising:
receiving, by the mobile application, raw data generated by one or more accelerometers being worn by the user;
processing, by the mobile application, the received raw data generated by the one or more accelerometers to generate usable data representing a particular motion and the number of repetitions of the motion being performed by the user; and
displaying, on the mobile application, an indication of the particular motion and the number of repetitions.

5. The method of claim 1, further comprising:
transmitting the raw data to one or more servers.

6. The method of claim 5, further comprising:
receiving, from the one or more servers, a pattern that commonly appears in raw data, the pattern identifying the occurrence of a condition; and
comparing the pattern to new raw data received from the one or more sensors to identify when the new data indicates that the user has experienced the condition.

7. The method of claim 6, wherein the condition comprises a health condition.

8. The method of claim 6, wherein the condition comprises a performance condition.

9. The method of claim 8, wherein the performance condition comprises one of the user's VO₂ max or lactate threshold.

10. The method of claim 6, further comprising:
in response to identifying that the user has experienced the condition, notifying the user of the occurrence of the condition.

11. The method of claim 6, further comprising:
receiving, by the mobile application, raw data generated by one or more accelerometers being worn by the user while experiencing the condition;
processing, by the mobile application, the received raw data generated by the one or more accelerometers to generate usable data representing a particular motion being performed by the user; and
correlating the occurrence of the condition with the particular motion.

12. The method of claim 11, wherein the usable data also represent a number of repetitions of the particular motion being performed or a rate at which the particular motion is being performed by the user, the method further comprising:
correlating the number of repetitions or rate with the occurrence of the condition.

13. The method of claim 1, further comprising:
identifying that the performance of at least one of the one or more sensors can be improved based on how the user is using the at least one sensor as indicated by the raw data; and
transmitting, to the at least one sensor a firmware update that customizes the performance of the at least one sensor to how the user is using the at least one sensor.

14. A system for monitoring physiological parameters during an activity using a mobile phone, the system comprising:
a mobile phone having an application for receiving raw sensor data from one or more sensors worn by a user while performing an activity;
the one or more sensors which detect one or more physiological parameters of the user while the user performs the activity;
wherein the one or more sensors transmit the raw sensor data to the mobile phone which processes the raw sensor data to generate usable data and to display the usable data on a display of the mobile phone.

15. The system of claim 14, wherein the one or more sensors comprise one or more of a pulse oximeter or a blood glucose monitor.

16. The system of claim 14, further comprising:
one or more accelerometers which generate raw data indicative of a movement performed by the user during the activity, wherein the mobile phone receives the raw data generated by the accelerometers and processes the raw data to determine a particular movement the user is performing.

17. The system of claim 16, wherein at least one accelerometer and at least one sensor are incorporated into a single device worn by the user.

18. The system of claim 17, wherein the single device comprises a bracelet.

19. The system of claim 17, wherein the single device comprises a shoe clip.

20. The system of claim 14, wherein the one or more sensors are incorporated into one or more devices worn by the user, and wherein each of the devices includes an accelerometer which generates raw data indicative of a movement performed by the body part to which the device is attached.
